(19) United States
(12) Patent Application Publication
(10) Pub. No.: US 2013/0156835 A1
(43) Pub. Date: Jun. 20, 2013

(54) NOVEL FORMULATIONS OF ACTIVE INGREDIENT(S) OF PLANT ORIGIN OR SYNTHETIC ANALOGUES THEREOF OR OF EXTRACT(S) OF PLANT ORIGIN CONTAINING SAME, AND OF LECITHIN

(71) Applicant: Alberto Sardo, Chateaurenard (FR)

(72) Inventor: Alberto Sardo, Chateaurenard (FR)

(21) Appl. No.: 13/641,429

(22) PCT Filed: Apr. 14, 2011
(86) PCT No.: PCT/FR11/50861

§ 371 Date: Feb. 28, 2013

(30) Foreign Application Priority Data
Apr. 16, 2010 (FR) 10 52903

(51) Int. Cl. A61K 47/24; A61K 36/287; A01N 65/28; A01N 25/02; A01N 65/12; A61K 36/61; A61K 31/085

(52) U.S. Cl. CPC A61K 47/24; A61K 36/61; A61K 36/287; A61K 31/085; A01N 25/02; A01N 65/12; A01N 65/28
USPC 424/405; 424/400; 424/725; 424/745; 514/68; 514/720

(57) ABSTRACT

The present invention relates to a composition comprising one or more active ingredients of plant origin, or their synthetic analogues or extract(s) of plant origin containing them, encapsulated by the lecithin in an aqueous solution.

The present invention relates to a novel formulation of active ingredient(s) encapsulated by lecithin, and to their uses in human or veterinary therapeutics, or in plant protection.

Active ingredients of plant origin and extracts of plant origin containing them generally have particularly attractive biological activity.

Thus, eugenol or clove oil which contains it, is known for its anti-germinating, fungicidal or anti-bacterial activity. Pyrethrum, as well as pyrethrins which may be extracted therefrom have remarkable insecticidal activity.

Nevertheless, these agents have formulation and application difficulties, notably related to their low solubility. Generally, it is impossible to prepare aqueous solutions therefrom; they thus require the use of solvents or emulsifiers for stabilizing the aqueous emulsions.

It is therefore desirable to make available novel formulations of such agents in an aqueous solution.

Thus, according to a first object, the present invention relates to a composition comprising one or more active ingredients of plant origin, or their synthetic analogues or extract(s) of plant origin containing them, in an aqueous solution, said composition comprising:

one or more active ingredients of plant origin or extracts of plant origin containing them, or synthetic analogues,

lecithin,

alcohol,

one or more sugars,

water,

such that the active ingredient(s) of plant origin or extract(s) of plant origin containing them, or synthetic analogue(s) are encapsulated by the lecithin.

The compositions according to the present invention are particularly advantageous in that said solution is stable, free of emulsifier.

According to a particular aspect, the composition according to the invention does not require any other ingredient. It may therefore essentially consist in the aforementioned ingredients.

As an extract of plant origin, mention may notably be made of essential oils such as clove oil, or thyme oil or extracts of plants, such as extracts of flowers, extracts of pyrethrum flowers, notably.

As an active ingredient contained in such extracts, mention may thus notably be made of eugenol, isoeugenol, thymol, carvacrol, pyrethrins, and notably pyrethrin 1, pyrethrin 2, jasmolin 1, jasmolin 2, cinerin 1, cinerin 2.

By [symbol] synthetic analogue [symbol] is meant active agents obtained by semisynthesis or hemisynthesis, for which the structure is similar or at least partly mimics the structure of active ingredients of plant origin.

As a synthetic analogue of said active ingredients, mention may notably be made of synthetic analogues of pyrethrins, such as synthetic pyrethroids such as bifenthrin, bioresmethrin, deltamethrin, depallethrin, ethofenprox, fenpropathrin, cypermetrin, fenvalerate, esfenvalerate, cyfluthrin, alphamethrin, tralomethrin, fluvalinate, permethrin, lambda-cyhalothrin, flucythrinate, tefluthrin, tralomethrin, zetacypermethrin, betacyfluthrin.

Advantageously, the active ingredient(s) of plant origin or extract(s) of plant origin containing them, or synthetic hydrophobic analogue(s) may be encapsulated by the compositions according to the invention.

As lecithin, mention may notably be made of natural or modified lecithin, preferably natural lecithin.

As a sugar, mention may notably be made of saccharose, glucose, fructose, sucrose and mannose.

As an alcohol, mention may notably be made of aliphatic alcohols and in particular ethanol.

Unexpectedly, it was discovered that in the presence of sugar, it was possible to obtain complete solubilization of lecithin and of the active ingredient, in a mixture of water and alcohol, thereby allowing a perfect coating by micro-encapsulation.

The compositions according to the invention allow the encapsulation of the active ingredient within lecithin, notably by lamellar encapsulation. Unlike the aqueous dispersions containing emulsifiers, the solutions according the invention withstand the [symbol] wash out [symbol] phenomenon by rain or biological fluids.

The compositions according to the invention therefore allow a longer duration of exposure of the active ingredient.

Generally, the composition according to the invention comprises:

from 5 to 30%, preferably 10 to 20% of active ingredient(s) of plant origin, extracts of plant origin containing them, or synthetic analogue(s) or mixtures thereof,

from 15 to 40%, preferably from 20 to 30% of lecithin,

from 10 to 55%, preferably 15 to 30% of alcohol;

from 10 to 35%, preferably 10 to 20% of one or more sugars, in solution in water.

According to another object, the present invention also relates to the method for preparing a composition according to the invention comprising:

1) preparing a solution of said sugar(s) in water;

2) adding to said solution the alcohol and the active ingredient(s) of plant origin or extract(s) of plant origin containing them, or synthetic analogue(s),

3) adding lecithin.

Generally, addition of lecithin is achieved with stirring, until complete dissolution of the lecithin.

According to another object, the present invention also relates to a method for encapsulating one or more active ingredients of plant origin or extract(s) of plant origin containing them or synthetic analogue(s) by the lecithin, comprising the application of the above method.

According to a first embodiment, mention may thus be made of the compositions comprising:

clove oil or eugenol,

lecithin,

alcohol,

one or more sugars,

in solution in water.

According to another embodiment, the present invention also relates to compositions comprising as an active ingredient, a pyrethrum extract or one or more natural pyrethrums or their synthetic analogues, in combination with eugenol or clove oil.

Said compositions then contain:

a pyrethrum extract, or one or more natural pyrethrums or synthetic pyrethroids,

lecithin,

alcohol,

one or more sugars,

water,

in solution.

Said compositions may further comprise eugenol or clove oil.

Generally, the compositions are such that the clove oil/pyrethrum (weight) ratio is comprised between 1 and 5.

According to another object, the present invention relates to the human or veterinary therapeutic use of a composition according to the invention comprising one or more active ingredients of plant origin or extract(s) of plant origin containing them or synthetic analogue(s) having therapeutic activity.

Said drug is intended for human or veterinary therapeutic use.

Thus, eugenol and clove oil have therapeutic activity and notably a bactericidal, fungicidal, anti-parasite activity. The compositions according to the invention containing them are thus particularly suitable for treating and/or preventing pathologies selected from intestinal parasitoses, mycoses, bacterial pathologies and/or pathologies related to unicellular organisms such as amoebas.

The drugs according to the invention comprise a composition according to the invention and optionally at least one pharmaceutically acceptable carrier. Said carrier may be selected depending on the contemplated applications. Thus, in the case when a skin application would be contemplated, they may be formulated with a formulation agent for topical administration, such as carriers for the formulation of cream, gel, pomade or lotion and notably cocoa butter, beeswax . . . such carriers are known per se and within the competence of one skilled in the art.

The compositions according to the invention may also be advantageously administered orally; they will generally appear as a solute or solution. They may optionally comprise sweetening agents and/or a flavor, if necessary.

The dosage may vary within the large limits (0.5 milligrams to 1,000 milligrams of active ingredients) depending on the therapeutic indication and on the administration route as well as on the age and weight of the subject.

According to another object, the present invention relates to the plant protective use of a composition according to the invention comprising one or more active ingredients of plant origin or extract(s) of plant origin containing them, or synthetic analogue(s) having a plant protective activity.

Thus, the pyrethrum extracts, pyrethrins and pyrethroids are known insecticides. The compositions according to the invention containing them are particularly suitable for insecticidal use.

On the other hand, pyrethrins or their synthetic analogues are generally used in combination with piperonyl butoxide as an adjuvant. Nevertheless, this compound has controversial secondary effects and is not of natural origin.

It was discovered that the compositions according to the invention comprising one or more pyrethrum extracts, pyrethrins and pyrethroids in combination with eugenol or clove oil have a highly interesting insecticidal activity, as compared with combinations of the aforementioned insecticides with piperonyl butoxide.

Notably for the use for insecticidal purposes, said compositions may either be as such, or be added to water so as to form a dispersion.

Generally, said dispersion is made by adding said composition to water, preferably with intensive stirring.

The present invention therefore also relates to said compositions, thereby obtained as a dispersion, comprising a composition according to the invention dispersed in water. Generally, the concentration of said dispersions is comprised between 0.5 and 10%.

According to another object, the present invention also relates to the method for insecticidal treatment comprising the application of the aforementioned compositions according to the invention. The method is particularly suitable for treating fruit and vegetables, generally in the orchard, plants or seeds.

Generally, the compositions according to the invention dispersed in water are more particularly suitable for application to orchards, on fruit and/or vegetables. Generally, this application may be carried out by means of sprinklers. The compositions as emulsions in water have the advantage of forming extremely fine particles, with an average diameter comprised between 40 and 80 micrometers. This small size thus allows the use of sprinklers with nozzles of small sizes, thereby allowing better application.

Generally, the compositions as such or dispersed in water may advantageously be used for application on grains, cereals or oilseeds, notably used for treating silos.

The following examples are given as an illustration and not as a limitation of the present invention.

### EXAMPLE 1

### Preparation of a Solution of Eugenol or Clove Oil

18 g of sugar are added to 19 g of water; after complete dissolution of the sugar, 21 g of ethanol followed by 17 g of clove oil are added; the mixture is stirred and 25 g of lecithin are added. The mixture is stirred until complete dissolution of the lecithin.

### EXAMPLE 2

### Preparation of a Pyrethrum and Clove Oil or Eugenol Solution

18 g of sugar are added to 20 g of water; after complete dissolution of the sugar, 21 g of ethanol followed by 4 g of a pyrethrum extract and 12 g of clove oil are added, the mixture is stirred and 25 g of lecithin are added. The mixture is stirred until complete dissolution of the lecithin.

### EXAMPLE 3

### Demonstration of the in Vivo Effect of a Composition According to the Invention in the Treatment of Amoebas

The composition of example 1 was administered to a human patient affected with amoebas at a dose of 10 g per day (5 g in the morning and 5 g in the evening), for three cycles with a duration of three weeks spaced apart, with two periods of three weeks.

The symptoms disappeared at the end of the treatment, the efficiency of the treatment was characterized by nanomicroscopic blood analysis.

### EXAMPLE 4

### Preparation of a Composition as a Dispersion in Water

The composition of Example 2 is added to the water at a 3% (weight) concentration, with stirring.

A dispersion is obtained. The size of the thereby obtained particles is comprised between 40 and 80 micrometers.

1-16. (canceled)

17. A composition of one or more active ingredients of plant origin or synthetic analogue(s) or extract(s) of plant origin containing said ingredient(s), in aqueous solution, said composition comprising:
from 5 to 30% of one or more active ingredients of plant origin or synthetic analogue(s) or extract(s) of plant origin containing said ingredient(s),
from 15 to 40% of lecithin,
from 10 to 52 of alcohol,
from 10 to 35% of one or more sugars,
water,
such that the active ingredient(s) of plant origin or synthetic analogue(s) or extract(s) of plant origin containing said ingredient(s) are encapsulated by the lecithin.

18. The composition according to claim 17 such that said solution is free of emulsifier.

19. The composition according to claim 17 or 18 essentially consisting in:
from 5 to 30% of one or more active ingredients of plant origin or synthetic analogue(s) or extract(s) of plant origin containing said ingredient(s),
from 15 to 40% of lecithin,
from 10 to 52 of alcohol,
from 10 to 35% of one or more sugars,
water.

20. The composition according to claim 17 or 18, such that the extract of plant origin is an essential oil.

21. The composition according to claim 17 or 18, such that the extract of plant origin is selected from clove oil, thyme oil and pyrethrum extracts.

22. The composition according to claim 17 or 18, such that said active ingredient(s) is(are) selected from eugenol, thymol, carvacrol, pyrethrins, pyrethroids and mixtures thereof.

23. The composition according to claim 17 or 18, comprising:
clove oil and/or eugenol,
lecithin,
alcohol,
one or more sugars,
water,
in solution.

24. The composition according to claim 17 or 18 comprising:
one or more natural pyrethrins or synthetic pyrethroids,
lecithin,
alcohol,
one or more sugars,
water,
in solution.

25. The composition according to claim 17 or 18 further comprising eugenol or clove oil.

26. A composition comprising a composition according to claim 24 as a dispersion in water.

27. A drug comprising a composition according to claim 17 or 18, comprising one or more active ingredients of plant origin or extract(s) of plant origin containing them, or synthetic analogue(s) having therapeutic activity with a pharmaceutically acceptable carrier.

28. The composition according to claim 17 or 18, comprising one or more active ingredients of plant origin or extract(s) of plant origin containing them or synthetic analogue(s) having a therapeutic activity, for treating and/or preventing in humans or animals, pathologies selected from intestinal parastisoses, mycoses, bacterial pathologies, pathologies related to unicellular organisms.

29. An insecticidal treatment method comprising the application of a composition according to claim 17 or 18, comprising one or more active ingredients of plant origin or extract(s) of plant containing them, or synthetic analogue(s) having a plant protective activity.

30. A method for preparing a composition according to claim 17 or 18 comprising:
1) preparing a solution of said sugar(s) in water;
2) adding to said solution the alcohol and the active ingredient(s) of plant origin or extract(s) of plant origin containing them, or synthetic analogue(s), and
3) adding lecithin.

31. A method for encapsulating one or more active ingredients of plant origin or extract(s) of vegetable origin containing them, or synthetic analogue(s), by the lecithin, comprising the method according to claim 30.

32. A composition comprising a composition according to claim 25 as a dispersion in water.

33. An insecticidal treatment method comprising the application of a composition according to claim 26 comprising one or more active ingredients of plant origin or extract(s) of plant containing them, or synthetic analogue(s) having a plant protective activity.

34. An insecticidal treatment method comprising the application of a composition according to claim 32 comprising one or more active ingredients of plant origin or extract(s) of plant containing them, or synthetic analogue(s) having a plant protective activity.
